SEQUENCE LISTING (38 sequences)

SEQ ID NO: 1 - DNA, 47 bp, Artificial Sequence
  shRNA for human beta2m transcript (NM_004048.2, start nt 244)
gaatggagagagaattgaattcaagagattcaattctctctccattc

SEQ ID NO: 2 - DNA, 47 bp, Artificial Sequence
  Less efficient shRNA sequence for human beta2m transcript HS_shN22m-2 (NM_004048.2, start nt 118)
gctatccagcgtactccaattcaagagattggagtacgctggatagc

SEQ ID NO: 3 - DNA, 51 bp, Artificial Sequence
  shRNA for human CIITA HS_shCIITA (NM_000246, start nt 400)
aagagaccagggaggcttatgttcaagagacataagcctccctggtctctt

SEQ ID NO: 4 - DNA, 49 bp, Artificial Sequence
  Less efficient shRNA for human CIITA HS_shCIITA-2 (NM_000246, start nt 3469)
gcccattcagtgtccaggaattcaagagattcctggacactgaatgggc

SEQ ID NO: 5 - DNA, 49 bp, Artificial Sequence
  Shorter less efficient shRNA for human CIITA HS_shCIITA-3 (NM_000246, start nt 400)
aagagaccagggaggcttatttcaagagaataagcctccctggtctctt

SEQ ID NO: 6 - DNA, 49 bp, Artificial Sequence
  shRNA sequence for swine (Sus Scrofa) beta2m SS_shN22m (L13854.1, start nt 3471)
cacgtgactctcgataagccttcaagagaggcttatcgagagtcacgtg

SEQ ID NO: 7 - DNA, 49 bp, Artificial Sequence
  Mismatched and less efficient shRNA sequence for swine (Sus Scrofa) 2m SS_shN22m-2 (L13854.1, start nt 3471)
cacgtgacactcgataagccttcaagagaggcttatcgagtgtcacgtg

SEQ ID NO: 8 - DNA, 49 bp, Artificial Sequence
  shRNA sequence for swine (Sus Scrofa) CIITA SS_shCIITA (XM_005662139.1, start nt1173)
ctgccacagtacgactttgtttcaagagaacaaagacgtactgtggcag

SEQ ID NO: 9 - DNA, 47 bp, Artificial Sequence
  shRNA sequence for rat (Rattus norvegicus) beta2m RN_N22m (NM_012512.2, start nt188 )
ggaaagaagataccaaatattcaagagatatttggtatcttctttcc

SEQ ID NO: 10 - DNA, 51 bp, Artificial Sequence
  shRNA sequence for rat (Rattus norvegicus) CIITA RN_shCIITA (AF251307.1, start nt 176)
ggatatggaaatggatgaagattcaagagatcttcatccatttccatatcc

SEQ ID NO: 11 - DNA, 245 bp, Artificial Sequence
  Elongation Factor short (EFS) promoter (driving expression of reporter gene)
tggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcg

SEQ ID NO: 12 - DNA, 1080 bp, Artificial Sequence
  EF1alpha promoter (driving expression of reporter gene)
catttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttaaactacgggatcttcgaaggcctaagcttacgcgcgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcggggactagtcatatgataatcaacctctggatta

SEQ ID NO: 13 - DNA, 19 bp, Artificial Sequence
  Tet-operator element
tccctatcagtgatagaga

SEQ ID NO: 14 - DNA, 220 bp, Artificial Sequence
  H1 promoter to drive the expression of the shRNAs
aattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 15 - DNA, 333 bp, Artificial Sequence
  U6 Sequence to drive the expression of the shRNAs (weaker)
gatcccgcgtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggcgcgcgctcggcgcgcggtcacgctccgtcacgtggtgcgttttgcctgcgcgtctttccactgggg

SEQ ID NO: 16 - DNA, 63 bp, Artificial Sequence
  T2A peptide encoding nucleic acid sequence
  CDS 1..63: /note=“T2A peptide” /transl_table=1
ggctccggcgagggcaggggaagtcttctaacatgcggggacgtggaggaaaatcccggccca

SEQ ID NO: 17 - DNA, 1497 bp, Artificial Sequence
  SS_CD55 (SS = Sus Scrofa = pig)
atgagtcccctgccgcggagcgcccccgcggtgaggcgcctaatgggcggacagacgccgccgccgctgctgctgctgctgctgctgctgtgtatcccggctgcgcagggtgactgcagccttccacccgatgtacctaatgcccaaccagatttgcgaggtcttgcaagttttcctgaacaaaccacaataacatacaaatgtaacaaaggctttgtcaaagttcctggcatggcagactcagtgctctgtcttaatgataaatggtcagaagttgcagaattttgtaatcgtagctgtgatgttccaaccaggctacattttgcatctcttaaaaagtcttacagcaaacagaattatttcccagagggtttcaccgtggaatatgagtgccgtaagggctataaaagggatcttactctatcagaaaaactaacttgccttcagaattttacgtggtccaaacctgatgaattttgcaaaaaaaaacaatgtccgactcctggagaactaaaaaatggtcatgtcaatataacaactgacttgttatttggcgcatccatctttttctcatgtaacgcagggtacagactagttggtgcaacttctagttactgttttgccatagcaaatgatgttgagtggagtgatccattgccagattgccaagaaatttctccaactgtcaaagccataccagctgttgagaaacccatcacagtaaattttccaggtaccaaagccctatcatctcctcagaaaccctccacagcaaatactctagctacagagttactaccaactcctcaggaacccaccacagtaaatgttccagatagtaaagccatatcatctcctcagaaaccctccacagtaaatactccagctacagacttactaccaactcctcaggaacccaccacagtaaatgttccagatagtaaagccatatcatcttctcagaaaccctccacagtaaatactccagctacagacttactaccaactcctcaggaacccaccacagtaaatgttccagatagtaaagccatatcatctcctcagaaaccctccacagtaaatactccagctacagacttactaccaactcctcaggaacccaccacagtaaatgttccagctccagaagttccatcaacttctcagaaaccttccacagcaaatgattctgcaccacaggccacatcaacaacccaaagattcaccacagcaaaagcttcactgacacagactcttcgagaaacaggaaagtccactcctgtacatacctcaaaaactaaaggtctacagcaaagattgacttctgctcatgttacagcaacaaagtatccagctattcccagggccacaacgagttttcattcaagtacatctaaaaatcgaggaaacccttcttcaggcatgagaatcatgtcgtctggacgtgcatgtgtaacactgacagttttgcttatggtgctagtaaccattggctag

SEQ ID NO: 18 - DNA, 1401 bp, Artificial Sequence
  RN_CD55 (RN = Ratus Norvegicus = Rat)
  CDS 1..1401: /note=“rat CD55” /transl_table=1
atgatccgcacgcgggcgccggggactcgggctccgccgccgccgccgctgctgctgtcgctgtcgctgctgctgctgctgctgtccccgactgtaagcggagactgcggcccacctccagacattcctaatgccacgccaaactcgggtaaacacaccaagtttgcccaccaaagccaagtgacatattcgtgtaataagggcttcaaacaaattccagacaagccaaacacagtggtctgccttgaaaatgaccaatggtctaactacgaaactttctgtaataaaacctgtagtgctccactgagactgaattatgcatccctcaaaaaggagtatcgtaacatgaattttttcccaattggcaccactgtggaatttgagtgtcggccaggatttcaaaaagtaacttcacgcacaggaaaatcaacttgccttgaggaattagtatggtctccagttgttgaattttgtaaaaaaagagcatgccctaatcctagagaactgaaaaatggtcacatcaacataccaactgacatattattcggatcagaaataatcttctcatgcgacaaagggtacaagctggtcggtgtaacttctacattctgttctattacggggaatgctgtggactgggatgacgtatttccagtgtgcacaacaattttttgtccagacccaccaaaaatcaataatggaagaattcgagaggaaagcgactcctacgcatatagacagtcggtgacctattcctgtgaccaaggcttcattctggttggaaattcttccgttcattgtactctgaacggtgaagtaggagaatggagtagtgcaccacccaagtgcatagagagacacaaggtcccaactaagaccccaccagaagttgatattccaagtacaatgcctcatgaatccaccttaattaattttccaagtacaagagccccactgtctcataaacccactacagttaaagttccagcaacacagcatgtacctgtttccaagacaacggtacgtcatccaacgagaacatctaaagacagaggagagtctaattcaggtggtgaccattttatatatggaaagtttgacccacagggagttaaaagaaattgccatcactgtgggagacatagtccatatttctggaagagaatcttctcttataactgcctgagaaaaactatcaaaatgcagtgcctatgggtgcctaagggcagatgggtaaagccttttaatcggctggttttttggacaccgttaggcatccagcatggtccttcatatccccaacagtgctcctgctctaaggcagtcttaggacagcttcgggtcatctggccaatcagtcaggtagcactgagtgcccgctgtgtcagtgtgtcattttag

SEQ ID NO: 19 - DNA, 372 bp, Artificial Sequence
  SS_CD59 of Sus scrofa coding sequence
atgggaagcaaaggagggttcattttgctctggctcctgtccatcctggctgttctctgccacttaggtcacagcctgcagtgctataactgtatcaacccagctggtagctgcactacggccatgaattgttcacataatcaggatgcctgtatcttcgttgaagccgtgccacccaaaacttactaccagtgttggaggttcgatgaatgcaatttcgatttcatttcgagaaacctagcggagaagaagctgaagtacaactgctgccggaaggacctgtgtaacaagagtgatgccacgatttcatcagggaaaaccgctctgctggtgatcctgctgctggtagcaacctggcacttttgtctctaa

SEQ ID NO: 20 - DNA, 381 bp, Artificial Sequence
  RN_CD59 rattus norvegicus coding sequence
  CDS 1..381: /note=“CD59 of rattus norvegicus” /transl_table=1
atgagagctcggaggggattcatcttactcctgcttctggctgtcctctgttccacaggtgttagcctcagatgctacaactgtttagacccggtttcttcatgcaaaacaaacagcacttgctctcctaacctggatgcttgtcttgttgctgtatccggaaagcaagtctatcaacagtgttggagattttcggattgtaatgccaagttcattttgagccgactagaaatcgcaaacgtacaatacagatgctgccaggcggacttgtgtaacaaaagcttcgaagacaagccaaacaatggggcaatctccctattggggaagacagcgttgctggtgacctcggttctggcggccattttgaagccttgtttctaa

SEQ ID NO: 21 - protein, 126 aa, Artificial Sequence
  [CDS]1..381 from SEQ ID NO 20
MRARRGFILLLLLAVLCSTGVSLRCYNCLDPVSSCKTNSTCSPNLDACLVAVSGKQVYQQCWRFSDCNAKFILSRLEIANVQYRCCQADLCNKSFEDKPNNGAISLLGKTALLVTSVLAAILKPCF

SEQ ID NO: 22 - protein, 363 aa, Artificial Sequence
  SS_CD46 of sus scrofa
MMAFCALRKALPCRPENPFSSRCFVEILWVSLALVFLLPMPSDACDEPPKFESMRPQFLNTTYRPGDRVEYECRPGFQPMVPALPTFSVCQDDNTWSPLQEACRRKACSNLPDPLNGQVSYPNGDMLFGSKAQFTCNTGFYIIGAETVYCQVSGNVMAWSEPSPLCEKILCKPPGEIPNGKYTNSHKDVFEYNEVVTYSCLSSTGPDEFSLVGESSLFCIGKDEWSSDPPECKVVKCPYPVVPNGEIVSGFGSKFYYKAEVVFKCNAGFTLHGRDTIVCGANSTWEPEMPQCIKDSKPTDPPATPGPSHPGPPSPSDASPPKDAESLDGGIIAAIVVGVLAAIAVIAGGVYFFHHKYNKKRSK

SEQ ID NO: 23 - protein, 349 aa, Artificial Sequence
  HS_CD46
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKGPRPTYKPPVSNYPGYPKPEEGILDSLDVWVIAVIVIAIGKQMVELNMPLTRLNQPLQQSREAE

SEQ ID NO: 24 - protein, 381 aa, Artificial Sequence
  CD55
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 25 - protein, 128 aa, Artificial Sequence
  CD59
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 26 - DNA, 360 bp, Artificial Sequence
  beta2microglobulin encoding sequence of rattus norvegicus
atggctcgctcggtgaccgtgatctttctggtgcttgtctctctggccgtcgtgcttgccattcagaaaactccccaaattcaagtgtactctcgccatccaccggagaatgggaagcccaacttcctcaactgctacgtgtctcagttccacccacctcaaatagaaattgagctactgaagaatggaaagaagataccaaatatcgagatgtcagatctgtccttcagcaaggactggtctttctacatcctggctcacactgaattcacacccaccgagaccgatgtatatgcttgcagagttaaacacgtcactctgaaggagcccaaaaccgtcacctgggaccgagacatgtaa

SEQ ID NO: 27 - DNA, 3159 bp, Artificial Sequence
  MHC class II transactivator (CIITA) rattus norvegicus encoding sequence
  CDS 1..3159: /note=“CIITA” /transl_table=1
atggagctggggtctccagagggcagctacctggagctccttaacagtgatgctgaccccctacacctctaccacctctatgaccagatggaccttgctggggaggaagagatcgaactcaactcagagacagacatggacaccatcaactgtgaccagttcagcaagctgttgcaggatatggaaatggatgaagagacccgggaggcctatgccaatatcgcggaactggatcagtacgtgttccaggatacccagctggagggcctgagcaaggacctcttcatacagcacattggaccagaggaaggctttggtgagaacgtagagggccctgcagaatcaggagagaagcctcagaagagacgcttcccagaagagcatgctatggactcaaagcacaggaaactagtgcctccctctaggacctcactgaactatttggatctccccgctgggcatatccagatcttcaccactctgccccagggactctggcaaatctcaggggttggcacagggctctccagtgtcctagtctaccacggtgagatgccccaggtcaaccaattactctcttcaagcagccccactgtccccagtctccccgagtccccagaccggcctggctccaccagccccttcgcgccatccacagctgacctgcccggcatgcctgaacctgctctgacctcccgtgtaaatgagacagaggacacaacacccccctccccatgccaagaggatcccgagccttccatcaagcttccaaaatggccagagtccgtggagcggttccagcactccctacaggacaagtaccaagcactgcccgggagcccaagtggtcctctggtagccatggagctggtgcgggccaggttggaaagaggtagcaacaagagtcaggaaagggagctggccactcctgactggacagagcgccagctagcccatggcggtctggccgaggtacttcgggttgtcggcgactccaggagatcacgagagacacaggtggtcgctgtgctgggcaaggctggtcagggcaagagccactgggccaggacagtgagccacatttgggcacaaggccagttaccacaatatgactttgtcttctacgtcccctgtcaatgcttggatcgccctggggacacctaccacctgcgggatctgctctgtcccccgaacctgcagccactggccatgaatgacgaggtcattgatcatattgtgaggcagccggaccgtgttctgctcatcctagatgcttttgaggagctagaggcccaagacagcctcctgcatggaccctgtggacctctgtccccagagccctgctccctccggggactgctggctgggctcttccagcgtaagctgctgcgaggctgcaccctgctcctcacagcccggccccggggccgcctggctcagagcctgagcaaggcagatgccatcttcgaggtgcccagcttctctacgaaacaggcccagatctatatgaggcactactttgagaactcagggacagcagggagtcacgacaaggccctgggcctcctggagggccagccttttctcctcacccacagtcacagccctgttctgtgcagtgccgtgtgtcagctctccaaggccctgctggatcagggcgcaggggcccagctaccttgcacactcacaggactctatgttggcctgctaggccctgcagctcagaacagtcctcctggagccttagttgagttggccaagctagcctgggagctgggacggaggcaccaaagcaccttgcaggaaacccagtttccatccgtggaagtgaagacctgggctgtggcccaaggcttgatgcagcagaccacagaggcccaactggctttctccagttttctgctacagtgcttcctgggtgctgtgtggctggcacagtgcaatgagatgaaagacaaggagctgccacagtacctggccttgaccccgaggaagaagcgaccctatgacaactggctggagggcgtaccacgctttctggctggcttagttttccagcctcgagcccactgcctgggagctctggtggagcctgcaatggctgcagtggcagataggaaacggaaggttcttaccaggtacctgaagcgcttgaagctggggacacttcgggccgggaggttgctggagctgctacactgtgcccacgaaacacaggaacctggtatttggaagcatgtttcacaccagctcccagggcacctctccttcctgggcacacggctcacagccccagatgtgtatgttctgggcagggccttggagacagccagccaggacttctccttggaccttcgtcagactggcatcgagccctccagactgggagacctcgtgggactcagctgtgtcaccagtttcagggcctccttgagtgacacgatggcattatgggagtccctacagcatcagggagaaacccagctactccaggcggcagaggagaagttcaccattgagccatttaaggccaaatccccaaaggatgtggaagacctggacagccttgtgcagacccagaggctgagaaacccctcagcggatgcagccaaggatcttcctgccatccgggaccttaagaagctagagtttgcattgggccccgtcttgggcccgcaggctttccccacactggccaagatccttccagcattctcttccctgcagcacctggacctggactcacttagcgagaacaagatcggggacaagggtgtgtcgaagctctcagccaccttccctcagctgaaggccctggagacgctcaacctgtcccagaatagcatcaccgatgtgggagcttgcaagcttgcagaagccctgccggccctggccaagtccctcctaaggctgagcttgtacaacaactgcatctgtgacgaaggggccaagagcctggcacgagtgcttccagacatggtgtccctccgggtgatggatgtccagttcaacaagttcacggctgtcggggcccagcagctgacctccagccttcagaagtgccctcaggtggagacactggcaatgtggacacccactatcccctttggggtccaggaacacctgcagcagctggatgcccggattagcctgagatga

SEQ ID NO: 28 - DNA, 360 bp, Artificial Sequence
  Homo sapiens N22m encoding sequence
  CDS 1..360: /note=“human beta2microglobulin” /transl_table=1
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 29 - DNA, 3393 bp, Artificial Sequence
  Homo sapiens CIITA encoding sequence
  CDS 1..3393: /note=“CIITA” /transl_table=1
atgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 30 - DNA, 357 bp, Artificial Sequence
  Sus scrofa N22-microglobulin encoding sequence
  CDS 1..357: /note=“swine beta2microglobulin” /transl_table=1
atggctcccctcgtggccttggtcctgctcgggctgctctcactgtctggcctggatgcggtcgcgcgtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggatcagtatagctgccgcgtgaagcacgtgactctcgataagcccaagatagttaagtgggatcgagaccactaa

SEQ ID NO: 31 - DNA, 2697 bp, Artificial Sequence
  SS_CIITA
  CDS 1..2697: /note=“SS_CIITA” /transl_table=1
atggagttggggcctctggagggtgggtacttggagcttctcaacagcagtgccgaccctctgcagctctaccacctctatgaccggatggacctggctggagaagaagagatcgagctctgctcagaacctgacacggacaccatcaactgcgaacagttcagcaggctgttgtgcgacatggaagcagatgaagaaaccagggaaacttacgccagtatcgcggaactggaccagtatgtttttcaagactctcagctggagggcctgggcaaagacattttcattgagcacataggattggaagaaatgatcagtgagagcgtggaggtgctggaggactcagggcggaaaagtcagaaaagatctttcccggaggagctgcctgcggatctgaagcacaggaagctagccgagcccctcgccatgcccatggtgactggcactttcctggtggggccagtgagcgactcctcagctcgaccctgcccatcacctcctgctctgttcaacaaggaatcaacacccagccaggcccagctggaggacgctgtcccaatgccggcgcccccttcaggttccttgttgagctgcctgagtgtccctgctggacctattcagatcatccccacgctctccaccctgccccaggggctctggcacatctcaggggccgggacaggggtctccagtatactcatctaccaaggtgagcgtgggaagccaggctccccaccccctctgcctgtgaccccggtcctgccactagcatcctcccctcccccactcccgcctttgacctgtgcagagactgtggagcagttccaccactcactccgggacaggtaccaagccaagcccgcaggcccggaaggcatcctggtggaggtggacctggtgagggtgcggctggagaggagcagcagcaagagtcaggagagagagctggcctccctggactgggcagagcggcagccagcccgagggggtctggcggaggtgctgctggccgctagcgaccgccaggggccacgcgagacgcaggtgatcgccgtgctcggcaaagcaggacaagggaagagtcactgggcccaggccgtgagctgggcctgggctgacggccagctgccacagtacgactttgtcttctgcatcccctgccactgtttggaccggccggggaacacctaccgcctgcaggatctgctcttctccctgggcccacagcccctgcccatggacgacgaggtcttcagttacatcttgaggcggccggaccgcgttctgctcatcctggatgccttcgaggagcgcgaagcccaggacggcttcgtgcacagcgcgggcggacccctgtcctcagaaccccgctcccttcgggggctgctggctgggctcctccagcgcaagctgctgcgaggctgcaccctgctgctcacggcccggccccggggccgcctggcccagagcctgagcaaggccgacgccctgtttgaggtggccggcttctccgcacagcaggccaagacctacatgctgcgctactttgagtgtcggggggcccgtgagcgccagaagagagccctggagctcctccaggcacagccgtttctcctgagtcacagccacagcccttccgtgtgccgggccgtgtgccggctctcagagaccctcctggagctgggcgaggaggcagagctgccctccacgctcaccggcctctacgtcggcctcctaggaccagcggcccgcgaaagccccccgggtgccctggtgggactggccagactggcctgggaactgggccgccgtcaccacagcagcttgcaggagggccagttcccatcggcagaggccagggcctgggctgtggcccaaggcttggtgcagcgtgccccgggggccccgggggcccctgagctggccttctccagcttcctcctgcagtgcttcctgggggccgtgtggctggctctgagcagcgagatcaaggacaaggagctgccgcagtatttggcattaacccctaggaagaagaggccctatgacaactggctggaggctgtgccacgctttctggtcgggctggtcttccagcctcgcgcccgctgcctgggagccctggcagggctggtggcagccaccttggcggaccggaagcagaaggtgctcaacaggtacctgaagcggctgcagcccgggaccctgcaggcagggcggctgctggagctgctgcactgcacgcacgaggccctggattctgggctttggcagcatgtgctgcaggggctcccgacccaactctcctttctgggcactcggctcacgcctccggacacccacgtgctgggcagcgccttggtggctgcaggccgagacttctccctggacctccgcagcactggcattgacccctctggactggggagcctcgtgggactcagctgtgtcacccatttcagggccgccttgagtgacacagtggggctgtgggagtctctacagcaacgtggggagaccaagctactccaggcactggaggagaaatttaccattgagcctttcaaggccaagtccatgaaggatgtggaagacctgggcaacctcgtgcagatccagaggacgagaagctcttctgaagacatggctggggaactccctgctgtccgggacctaaagaagttggaatttgcgtga

SEQ ID NO: 32 - protein, 21 aa, Artificial Sequence
  [CDS]1..63 from SEQ ID NO 16
GSGEGRGSLLTCGDVEENPGP

SEQ ID NO: 33 - protein, 466 aa, Artificial Sequence
  [CDS]1..1401 from SEQ ID NO 18
MIRTRAPGTRAPPPPPLLLSLSLLLLLLSPTVSGDCGPPPDIPNATPNSGKHTKFAHQSQVTYSCNKGFKQIPDKPNTVVCLENDQWSNYETFCNKTCSAPLRLNYASLKKEYRNMNFFPIGTTVEFECRPGFQKVTSRTGKSTCLEELVWSPVVEFCKKRACPNPRELKNGHINIPTDILFGSEIIFSCDKGYKLVGVTSTFCSITGNAVDWDDVFPVCTTIFCPDPPKINNGRIREESDSYAYRQSVTYSCDQGFILVGNSSVHCTLNGEVGEWSSAPPKCIERHKVPTKTPPEVDIPSTMPHESTLINFPSTRAPLSHKPTTVKVPATQHVPVSKTTVRHPTRTSKDRGESNSGGDHFIYGKFDPQGVKRNCHHCGRHSPYFWKRIFSYNCLRKTIKMQCLWVPKGRWVKPFNRLVFWTPLGIQHGPSYPQQCSCSKAVLGQLRVIWPISQVALSARCVSVSF

SEQ ID NO: 34 - protein, 1052 aa, Artificial Sequence
  [CDS]1..3159 from SEQ ID NO 27
MELGSPEGSYLELLNSDADPLHLYHLYDQMDLAGEEEIELNSETDMDTINCDQFSKLLQDMEMDEETREAYANIAELDQYVFQDTQLEGLSKDLFIQHIGPEEGFGENVEGPAESGEKPQKRRFPEEHAMDSKHRKLVPPSRTSLNYLDLPAGHIQIFTTLPQGLWQISGVGTGLSSVLVYHGEMPQVNQLLSSSSPTVPSLPESPDRPGSTSPFAPSTADLPGMPEPALTSRVNETEDTTPPSPCQEDPEPSIKLPKWPESVERFQHSLQDKYQALPGSPSGPLVAMELVRARLERGSNKSQERELATPDWTERQLAHGGLAEVLRVVGDSRRSRETQVVAVLGKAGQGKSHWARTVSHIWAQGQLPQYDFVFYVPCQCLDRPGDTYHLRDLLCPPNLQPLAMNDEVIDHIVRQPDRVLLILDAFEELEAQDSLLHGPCGPLSPEPCSLRGLLAGLFQRKLLRGCTLLLTARPRGRLAQSLSKADAIFEVPSFSTKQAQIYMRHYFENSGTAGSHDKALGLLEGQPFLLTHSHSPVLCSAVCQLSKALLDQGAGAQLPCTLTGLYVGLLGPAAQNSPPGALVELAKLAWELGRRHQSTLQETQFPSVEVKTWAVAQGLMQQTTEAQLAFSSFLLQCFLGAVWLAQCNEMKDKELPQYLALTPRKKRPYDNWLEGVPRFLAGLVFQPRAHCLGALVEPAMAAVADRKRKVLTRYLKRLKLGTLRAGRLLELLHCAHETQEPGIWKHVSHQLPGHLSFLGTRLTAPDVYVLGRALETASQDFSLDLRQTGIEPSRLGDLVGLSCVTSFRASLSDTMALWESLQHQGETQLLQAAEEKFTIEPFKAKSPKDVEDLDSLVQTQRLRNPSADAAKDLPAIRDLKKLEFALGPVLGPQAFPTLAKILPAFSSLQHLDLDSLSENKIGDKGVSKLSATFPQLKALETLNLSQNSITDVGACKLAEALPALAKSLLRLSLYNNCICDEGAKSLARVLPDMVSLRVMDVQFNKFTAVGAQQLTSSLQKCPQVETLAMWTPTIPFGVQEHLQQLDARISLR

SEQ ID NO: 35 - protein, 119 aa, Artificial Sequence
  [CDS]1..360 from SEQ ID NO 28
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 36 - protein, 1130 aa, Artificial Sequence
  [CDS]1..3393 from SEQ ID NO 29
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 37 - protein, 118 aa, Artificial Sequence
  [CDS]1..357 from SEQ ID NO 30
MAPLVALVLLGLLSLSGLDAVARPPKVQVYSRHPAENGKPNYLNCYVSGFHPPQIEIDLLKNGEKMNAEQSDLSFSKDWSFYLLVHTEFTPNAVDQYSCRVKHVTLDKPKIVKWDRDH

SEQ ID NO: 38 - protein, 898 aa, Artificial Sequence
  [CDS]1..2697 from SEQ ID NO 31
MELGPLEGGYLELLNSSADPLQLYHLYDRMDLAGEEEIELCSEPDTDTINCEQFSRLLCDMEADEETRETYASIAELDQYVFQDSQLEGLGKDIFIEHIGLEEMISESVEVLEDSGRKSQKRSFPEELPADLKHRKLAEPLAMPMVTGTFLVGPVSDSSARPCPSPPALFNKESTPSQAQLEDAVPMPAPPSGSLLSCLSVPAGPIQIIPTLSTLPQGLWHISGAGTGVSSILIYQGERGKPGSPPPLPVTPVLPLASSPPPLPPLTCAETVEQFHHSLRDRYQAKPAGPEGILVEVDLVRVRLERSSSKSQERELASLDWAERQPARGGLAEVLLAASDRQGPRETQVIAVLGKAGQGKSHWAQAVSWAWADGQLPQYDFVFCIPCHCLDRPGNTYRLQDLLFSLGPQPLPMDDEVFSYILRRPDRVLLILDAFEEREAQDGFVHSAGGPLSSEPRSLRGLLAGLLQRKLLRGCTLLLTARPRGRLAQSLSKADALFEVAGFSAQQAKTYMLRYFECRGARERQKRALELLQAQPFLLSHSHSPSVCRAVCRLSETLLELGEEAELPSTLTGLYVGLLGPAARESPPGALVGLARLAWELGRRHHSSLQEGQFPSAEARAWAVAQGLVQRAPGAPGAPELAFSSFLLQCFLGAVWLALSSEIKDKELPQYLALTPRKKRPYDNWLEAVPRFLVGLVFQPRARCLGALAGLVAATLADRKQKVLNRYLKRLQPGTLQAGRLLELLHCTHEALDSGLWQHVLQGLPTQLSFLGTRLTPPDTHVLGSALVAAGRDFSLDLRSTGIDPSGLGSLVGLSCVTHFRAALSDTVGLWESLQQRGETKLLQALEEKFTIEPFKAKSMKDVEDLGNLVQIQRTRSSSEDMAGELPAVRDLKKLEFA

(19) United States
(12) Patent Application Publication
(10) Pub. No.: US 2023/0010683 A1
(43) Pub. Date: Jan. 12, 2023

(54) TISSUE FOR USE AS ALLOGENEIC OR XENOGENEIC TRANSPLANT AND METHOD FOR ITS PRODUCTION

(71) Applicant: IMUSYN GMBH, Hannover (DE)

(72) Inventors: Rainer Blasczyk, Isernhagen (DE); Constanca Ferreira De Figueiredo, Neustadt am Rübenberge (DE)

(21) Appl. No.: 17/623,704

(22) PCT Filed: Jul. 6, 2020
(86) PCT No.: PCT/EP2020/068988

§ 371 Date: Dec. 29, 2021

(30) Foreign Application Priority Data
Jul. 4, 2019 (EP) 19184380.4

(51) Int. Cl. A61L 27/36; C12N 15/113

(52) U.S. Cl. CPC A61L 27/36; C12N 15/113; C12N 2310/14

(57) ABSTRACT

Tissue for use as a transplant, which tissue is allogeneic or xenogeneic and respectively the tissue may express an MHC I molecule that is immunologically incompatible to the transplant recipient and/or may express an MHC II molecule immunologically incompatible to the transplant recipient. The tissue suitable for use as a transplant and the method for its production include a genetic alteration of the tissue that provides for immunologic compatibility of the tissue with a transplant recipient. In the tissue for use as a transplant, which tissue expresses allogeneic or xenogeneic MHC I and/or allogeneic or xenogeneic MHC II molecules, the expression of the allogeneic or xenogeneic MHC I is downregulated by at least 50% to up to 90%, preferably the expression of the allogeneic or xenogeneic MHC I is downregulated by at least 60%.

The present invention relates to a tissue for use as a transplant and to a method of treatment by transplanting a tissue, which tissue is allogeneic or xenogeneic and respectively the tissue may express an MHC I molecule that is immunologically incompatible to the transplant recipient and/or may express an MHC II molecule immunologically incompatible to the transplant recipient. The tissue may alternatively express identical MHC I and MHC II molecules, as even identical MHC molecules in twins can present different peptides, which in transplantation result in minor histocompatibility antigens. The tissue suitable for use as a transplant and the method for its production comprise a genetic alteration of the tissue that provides for immunologic compatibility of the tissue with a transplant recipient. Further, the invention relates to nucleic acid constructs, e.g. viral vectors, suitable for introducing the genetic alterations into allogeneic or xenogeneic tissue in order to make them immunologically compatible to a transplant recipient.

The tissue comprising the genetic alteration according to the invention, and respectively tissue produced according to the invention, in spite of being allogeneic or in spite of the expression of allogeneic or xenogeneic MHC I and MHC II molecules has the advantage of being immunologically tolerated by the transplant recipient, and that therefore no or a reduced general immune suppression of the transplant recipient is required in order to avoid cellular and/or humoral transplant rejection (host-versus-graft (HvG) disease). A further advantage of the tissue according to the invention is that the immune system of the transplant recipient does not need to be modulated in the same extent as usually required, e.g. in an allotransplantation setting, e.g. by using powerful immunosuppressants. Accordingly, the genetically altered tissue according to the invention is suitable for use as a transplant in a recipient without temporary or permanent standard immune suppression, e.g. without or with reduced immunosuppressant for temporary or for permanent use in the treatment of adverse immune reactions, e.g. in the treatment of adverse immune reactions against the transplant, especially HvG disease. Preferably, the genetically altered tissue according to the invention is for use as a transplant with reduced or without general or specific immune suppression, e.g. excluding pharmaceutical compounds, e.g. immunosuppressants, for use in suppressing immunological reactions, e.g. for suppressing HvG disease, wherein the use in suppressing immunological reactions is preferably temporary, more preferably permanent.

### State of the Art

It is known that graft rejection by the recipient of the grafted transplant is a complex multifactorial process that results in the destruction of the transplanted tissue by the immune response of the recipient. This immune response is led by T-cells that directly or indirectly recognise MHC molecules of the transplant as allogeneic or xenogeneic. Also, B-cells are activated which leads to the production of antibodies which usually are directed against the immunologically incompatible MHC molecules of the transplant.

The tissue that is transplanted can easily be immunologically incompatible in respect of the recipient due to differences in its MHC class I or MHC class II antigens and/or in its allogeneic or xenogeneic peptides presented in even identical MHC molecules. A standard practice for reducing the immune reaction of the transplant recipient against the transplant (HvG disease) is a general and unspecific immune suppression of the transplant recipient, which in turn leads to severe side effects and to frequent occurrence of e.g. infections and increased risk of tumours.

EP 1546174 B1 describes generally that nucleic acid constructs encoding short hairpin RNA upon expression in a cell specifically suppress the expression of a hybridizing target nucleic acid.

WO 2018/215571 Al describes a method for genetically modifying a vascularized tissue by an ex vivo method in order to genetically modify the vascularized tissue, which may be an organ, for use as a transplant.

### Object of the Invention

It is an object of the invention to provide a tissue that expresses immunologically incompatible or compatible MHC I and/or immunologically incompatible or compatible MHC II molecules for use as a transplant in a future transplant recipient, which tissue is tolerated by the recipient. A further object is to provide an ex vivo method for manipulating an allogeneic or xenogeneic tissue to produce a tissue that is immunologically tolerated by a future transplant recipient and is therefore suitable for use as a transplant. A further object is to provide compounds, e.g. nucleic acid constructs, preferably packaged in viral particles, which are suitable for the manipulation of the allogeneic or xenogeneic tissue in order to make the tissue immunologically tolerable.

### Description of the Invention

The invention achieves the objects by the features of the claims, and specifically provides nucleic acid constructs which are suitable as compounds for the treatment of HvG disease, e.g. for making allogeneic or xenogeneic tissue immunologically tolerable, especially tolerable for a recipient of the allogeneic or xenogeneic tissue. Further, the invention provides allogeneic or xenogeneic tissue, e.g. tissue expressing an MHC I molecule and/or an MHC II molecule, which is genetically manipulated to make the tissue tolerable to the immune system of a recipient, and therefore the invention provides the genetically manipulated tissue for use as a transplant. Further, the invention provides an in vitro process for producing the tissue for use as a transplant.

Herein, tissue expressing an MHC I molecule and/or an MHC II molecule having immunologically detectable differences from the MHC molecules or the peptides presented therein of a recipient, and/or which MHC I molecule and/or MHC II molecule or the peptides presented therein would be recognized as non-self by a recipient and would elicit an immune reaction against the MHC I molecule and/or MHC II molecule, is referred to as allogeneic or xenogeneic.

Further, the invention provides a method of treatment of a patient, herein also referred to as a recipient of a transplant, by transplanting a tissue which according to the invention is suitable for use as a transplant, into the patient.

In transplant recipients, it was found that the downregulation of allogeneic MHC I molecule expression abrogates the generation of cytotoxic T-cells when the expression of the MHC I molecule is suppressed by at least 60% or by at least 70% or by at least 80% in comparison to wild-type cells, leaving a residual expression of at most 40%, respectively of at most 30% or of at most 20%. However, it was also found that the downregulation of the expression of allogeneic MHC I induces the generation or activation of cytotoxic NK-cells, e.g. when the suppression of the allogeneic MHC I molecule is by at least 80%, e.g. by 80 to 90%, compared to wild-type cells, leaving a residual expression of 20 to 10%.

The downregulation of expression of allogeneic MHC II molecules by at least 70%, e.g. by 70% up to at least 80% or more, leaving a residual expression of at most 30%, e.g. 30 to 20% or less, in comparison to expression of MHC II in wild-type cells, in transplant recipients was found to be sufficient to support the expansion of regulatory T-cells (Treg cells) of the recipients, e.g. in contrast to HLA-DR knockout cells which are not able to induce the expansion Treg cells.

In respect of antibody-mediated cytotoxicity, it was found in transplant recipients that a downregulation of the expression of allogeneic MHC I and of the allogeneic MHC II molecules by at least 70% or by at least or by at least 90%, leaving a residual expression of at most 30% or 20% or 10%, compared to wild-type cells, abrogates antibody-mediated cytotoxicity, even though antibodies directed against the MHC were generated.

It was found further in transplant recipients that the downregulation of the expression of allogeneic MHC I and of allogeneic MHC II by about 100%, i.e. to about 0% residual expression, compared to wild-type cells, abrogates formation of antibodies directed against the MHC I and of antibodies directed against the MHC II.

In order to provide for immunologic tolerance of a tissue for use as a transplant, which tissue expresses allogeneic or xenogeneic WIC I and/or allogeneic or xenogeneic WIC II molecules, which tolerance includes avoidance and prevention of an adverse immune reaction by a transplant recipient, the tissue of the invention is genetically manipulated, e.g. to quantitatively balance the level of expression of the allogeneic or xenogeneic MHC I and/or allogeneic or xenogeneic WIC II molecules, and to prevent a complement reaction.

The invention provides a tissue for use as a transplant, which tissue expresses identical or allogeneic or xenogeneic WIC I and/or identical or allogeneic or xenogeneic MHC II molecules,

wherein the expression of the identical or allogeneic or xenogeneic WIC I is downregulated by at least 50% to up to 90%, leaving a residual expression of at maximum 50% to 10%, wherein preferably the expression of the identical or allogeneic or xenogeneic WIC I is downregulated by at least 60%, more preferably by at least 70%, more preferably by 80% up to 90% or up to 85%, leaving a residual expression of the allogeneic or xenogeneic WIC I of at maximum 40%, at maximum 30%, more preferably at maximum 20% to 10% or 20% to 15%,

and wherein the expression of the identical or allogeneic or xenogeneic WIC II is downregulated by at least 50%, e.g. by at least 60%, e.g. downregulated by up to 90%, preferably downregulated by at least 70%, more preferably downregulated by at least 80%, e.g. downregulated by up to 90% or by up to 85%, leaving a residual expression of the identical or allogeneic or xenogeneic MHC II of at maximum 50%, e.g. of at maximum 40%, e.g. of at least 10%, preferably of at maximum 30%, more preferably of at maximum 20%, e.g. a residual expression of at least 10% or at least 15%,

wherein the downregulation of the identical or allogeneic or xenogeneic MHC I and/or of the identical or allogeneic or xenogeneic WIC II is in relation to its expression in wild-type tissue,

and additionally expressing at least one complement inhibitory molecule on the tissue, which complement inhibitory molecule is at least one of CD46, CD55 and CD59, preferably two of these, more preferably expression of CD46 and of CD55 and of CD59. Generally preferred, in the tissue for use as a transplant, which tissue expresses WIC I and MHC II molecules, the expression of the WIC I is downregulated by at least 50% to up to 90% and the expression of WIC I is downregulated by at least 60%. Herein, the term allogeneic WIC I and MHC II may comprise identical MHC I and identical MHC II, respectively.

Herein, the upper and lower percentages can be combined freely to define ranges of downregulation, respectively of residual expression.

Herein, expression in wild-type is the level of expression in the tissue prior to genetic manipulation, or the level of expression by the transplant recipient's own tissue, e.g. autologous tissue of the transplant recipient. Alternatively, e.g. using expression cassettes encoding inhibitory RNA for downregulation of MHC transcripts, downregulation can be determined as reduction of transcript levels, preferably normalized to transcript levels of a household gene. Transcript levels can quantitatively be determined by RT-PCR, allowing the determination of residual transcript levels that remain after hybridization with inhibitory RNA, and residual transcript levels are a measure for expression levels.

Upon transplantation into a recipient, the tissue according to the invention does not abrogate the activation and expansion of Treg-cells and prevents or avoids the generation or activation of cytotoxic T-cells, of cytotoxic NK-cells and delays or prevents or avoids the formation of antibodies originating from B-cells expressing antibody directed against the allogeneic or xenogeneic MHC I and/or directed against the allogeneic or xenogeneic MHC II.

Accordingly, the downregulation of the expression of the allogeneic or xenogeneic MHC I and/or of the allogeneic or xenogeneic MHC II in the tissue provides for tolerance by the immune system of the recipient of the tissue or mitigates the immune reaction against the tissue.

As residual expression of MHC molecules has been found to be essential to prevent NK cell-mediated responses and support Treg cell activity, the formation of antibodies may not completely be prevented. The expression of complement inhibitors was found to inhibit the cytotoxic effect of antibodies directed against the allogeneic or xenogeneic MHC I or against the allogeneic or xenogeneic MHC II molecules efficiently, e.g. co-expression of complement inhibitors CD55 and CD59, or co-expression of CD46 and CD55, or co-expression of CD46 an CD59, preferably of CD46 and of CD55 and of CD59 in the tissue, only or particularly when the expression of the allogeneic or xenogeneic MHC I and of the allogeneic or xenogeneic MHC II is downregulated.

Expression levels of WIC II and of WIC I can be determined e.g. by FACS using antibodies directed against MHC II, respectively against MHC I, using isotype controls as reference to determine the background fluorescence. Transcripts levels of MHC II and of MHC I can be determined e.g. by Real-Time PCR using primers and probes directed against MHC II, respectively against MHC I, preferably using expression of a household gene as an internal standard and normalizing expression of MHC II, respectively of WIC I, to the expression of the household gene. A suitable household gene is e.g. beta-actin, the expression of which can be determined for normalizing expression of WIC II and of WIC I, in wild-type tissue and in tissue genetically manipulated with nucleic acid constructs that downregulate expression of WIC II and of MHC I, respectively.

The MHC I and the MHC II of the tissue is allogenic or xenogeneic in respect of the transplant recipient. For example, the MHC which is xenogeneic can be a swine leukocyte antigen (SLA) for a non-swine recipient, e.g. for a human recipient, and an allogeneic MHC can be a human leukocyte antigen (HLA) which differs from the HLA type of the human recipient and/or differs by presenting allogeneic peptides from the recipient even if the donor and recipient have the same HLA type.

It was found that the balanced downregulation of the allogeneic or xenogeneic MHC I expression by at least 60% of the expression level compared to wild-type expression, and by at maximum 90%, leaving a residual expression of at least 40% to 10%, compared to expression of the wild-type, in combination with a downregulation of the expression of the allogeneic or xenogeneic MHC II by at least 50% up to 80%, leaving a residual MHC II expression of at least 50% to 20%, compared to expression of the wild-type, significantly reduces the interaction of cytotoxic T-cells, e.g. via their T-cell receptor (TCR), but enables the interaction of the Treg cells via their TCR with MHC II, and enables the interaction of NK-cells with MHC Ito a sufficient extent to prevent cytotoxicity by NK-cells. The residual expression of the allogeneic or xenogeneic MHC I and of the allogeneic or xenogeneic MHC II results in generation of antibody producing cells, which antibodies are directed against the MHC I and/or against the MHC II. The expression of at least one molecule, preferably of two or of all three molecules, selected from CD46, CD55 and CD59, on the tissue has been found to prevent detrimental effects of these antibodies, e.g. to provide protection against complement activity against the tissue.

In addition, the tissue can be genetically manipulated to downregulate expression of gene products other than MHC I and MHC II, which gene products are foreign to the transplant recipient, e.g. downregulated by at least 80%, preferably by at least 90%, more preferably downregulated by 100%.

For downregulation of the expression of the allogeneic or xenogeneic MHC I and of the allogeneic or xenogeneic MHC II, as well as for expression of at least one of the complement inhibitory molecules the tissue can be genetically manipulated by integration of nucleic acid constructs which encode inhibitory RNA sequences, e.g. siRNA, preferably short hairpin RNA (shRNA), which downregulate the expression of MHC class I and MHC class II genes, e.g. by being specific for the allogeneic or xenogeneic MHC I gene transcripts, respectively for the allogeneic and/or xenogeneic MHC II gene transcripts. Accordingly, in the tissue the expression of the allogeneic or xenogeneic MHC I and/or the expression of the allogeneic or xenogeneic MHC II is downregulated by the tissue being genetically manipulated to contain at least one nucleic acid construct comprising at least one expression cassette encoding inhibitory RNA interfering with at least one transcript encoding the allogeneic or xenogeneic MHC I molecule and/or encoding the allogeneic or xenogeneic MHC II molecule.

For downregulation of MHC I expression, the inhibitory RNA, e.g. siRNA, preferably shRNA is preferably specific for the transcript of the 02-microglobulin gene (β2m), and/or specific for the transcript of at least one, or two, preferably for transcripts of all three heavy chains of e.g. HLA-A, HLA-B and HLA-C molecules or SLA-1, SLA-2 and SLA-3 molecules.

For downregulation of MHC II expression, the inhibitory RNA sequences preferably are specific for transcripts of at least one of the alpha chain and/or beta chain of e.g. HLA-DR, HLA-DQ and HLA-DP or SLA-DR and SLA-DQ, and/or by encoding inhibitory RNA sequences which are specific for the transcripts of the class II transactivator gene (CIITA), e.g. shRNA.

Generally, inhibitory RNA sequences can comprise one or more RNA sequences which are specific for one or more different transcripts.

In order to downregulate as described herein, the inhibitory RNA sequences, e.g. shRNA sequences, can have sequences which only suboptimally hybridize, e.g. by being less specific for the transcript, by designing the inhibitory RNA for hybridizing to less effective regions of the transcripts than SEQ ID NO: 1, which optimally hybridizes to the mRNA of shβ2m and may inhibit higher than desired in the invention, or SEQ ID NO: 3, which optimally hybridizes to the mRNA of CIITA, e.g. SEQ ID NO: 2 (HS shβ2m-2), which according to the invention less efficiently hybridizes to the mRNA of β2m, leading to a HLA class I suppression of only up to 60%, or SEQ ID NO: 4 (HS shCIITA-2), which according to the invention less efficiently hybridizes to the mRNA of CIITA, leading to a HLA class II expression of only up to 60%, by controlling transcription of the inhibitory RNA sequences by using a promoter having an activity to express the inhibitory RNA only at the activity required for the downregulation, e.g. by using the U6 sequence, SEQ ID NO: 15, causing a 10%-20% weaker expression of the shRNAs than e.g. a H1 promoter, having SEQ ID NO: 14, or by the inhibitory RNA sequences being shorter than the optimal inhibitory RNA, e.g. SEQ ID NO: 5 for human CIITA (HS shCIITA-3) , or by the inhibitory RNA having one or more mismatches to the mRNA encoding MHC I or MHC II, e.g. one or more mismatches in the hybridizing section, e.g. according to the invention SEQ ID NO: 7 (SS_shβ2m-2) for the Sus scrofa β2m transcript. The efficacy of hybridization of inhibitory RNA to a transcript of the allogeneic or xenogeneic MHC I and/or MHC II molecules can be determined, e.g. by calculating or experimentally determining the stability of the hybrid, resp. determining the efficacy of RNA interference, e.g. by RT-PCR or flow cytometry. The human mRNA sequence encoding β2m is SEQ ID NO: 28, the human mRNA sequence encoding CIITA is SEQ ID NO: 29.

The design of a less effective inhibitory RNA, e.g. a shRNA, can be performed by in vitro experiments and by the support of RNAi designer software tools, e.g. by the publicly accessible tool BLOCK-iT™ RNAi Designer at

https://rnaidesigner.thermofisher.com/rnaiexpress.

Alternatively or additionally, the expression of the inhibitory RNA can be controlled by a conditional promoter system which can be regulated in a dose-dependent manner by the presence of a synthetic compound, e.g. using the tetracycline-inducible promoter or a tetracycline-repressible promoter for controlling the transcription of inhibitory RNA, which is induced, resp. repressed, by administration of tetracycline.

Alternatively or additionally, the expression of MHC class I or MHC class II can be downregulated by modifying the original promoter, e.g. by causing an activity lower than the activity of the wild-type promoter controlling expression of the allogeneic or xenogeneic MHC I or of the allogeneic or xenogeneic MHC II, respectively, or the splice sites of the individual MHC genes using genome editing approaches, e.g. CRSPR/Cas technology. In HLA-A*02:01 it has been shown that the nucleotide exchange from T to C at the genomic position −101 (g-101T>C) causes a decrease in the promoter activity leading to a low expression of this allele. In HLA-B*39:01 it has been shown that the nucleotide deletion of TC at the genomic positions −151-152 (g-151-152delTC) causes a decrease in promoter activity and low expression of this allele. In HLA-A*24:92 it has been shown that the nucleotide exchange from G to A at genomic position 708 (g708G>A) causes a low expression of this allele due to an insufficient splicing. Likewise, genome editing of other promoter positions and splice sites at the intron-exon borders are capable of substantially lowering the MHC expression without abrogating expression.

For expression of complement inhibitory molecules, e.g. at least one of CD46 (SEQ ID NO: 22, SEQ ID NO: 23), CD55 (SEQ ID NO: 17, SEQ ID NO: 18, SEQ ID NO: 24) and CD59 (SEQ ID NO: 19, SEQ ID NO: 20, SEQ ID NO: 25), sequences encoding these molecules can be contained in expression cassettes, preferably under the control of a constitutive promoter. The coding sequences for complement inhibitory molecules may comprise signal peptides resulting in expression on the cell surface of the tissue.

Exemplary promoters for controlling expression of the complement inhibitory molecules are e.g. the H1 promoter (SEQ ID NO: 14), the U6 promoter (SEQ ID NO: 15), the elongation factor short (EFS) promoter (SEQ ID NO: 11), and the EF1 alpha promoter (SEQ ID NO: 12). The nucleic acid constructs for expressing inhibitory RNA directed against the allogeneic or xenogeneic MHC I and for inhibitory RNA directly against the allogeneic or xenogeneic MHC II of the tissue, and expression cassettes for expression of at least one of the complement inhibitory molecules can be contained in different nucleic acid molecules, and preferably are contained on one common nucleic acid molecule, e.g. on a DNA or RNA contained in a viral vector, which preferably is packaged in a viral particle. Preferably, the viral vector is a lentiviral vector, packaged in lentiviral particles.

The tissue can be cells, e.g. single cells, e.g. islet cells, a portion of an organ or a complete organ, which organ is e.g. a kidney, liver, heart, lung, pancreas, skin, or an extremity.

In an embodiment, the tissue of the invention can be generated by genetically manipulating a non-human mammalian animal to contain expression cassettes for downregulating expression of MHC I and MHC II, and preferably for expressing at least two of the complement inhibitory molecules. Such an animal preferably is a swine. Also in this embodiment, the expression of inhibitory RNAs for downregulating transcripts of MHC I and MHC II can be controlled by a promoter which is inducible or repressible by a synthetic compound, e.g. a tetracycline-inducible promoter, especially the Tet-on or Tet-off promoter. Accordingly, such animals can be genetically manipulated, e.g. in their germline or by somatic genetic manipulation, e.g. by viral transduction or by use of CRISPR/Cas-based methods, to contain expression cassettes encoding inhibitory RNA for downregulating MHC I and MHC II, and optionally to contain expression cassettes for expression of at least two complement inhibitory molecules, which expression cassettes contain promoters that are inducible or are repressible by a synthetic compound. Such animals preferably are transgenic for these expression cassettes. For such animals that in their germline contain the expression cassettes, integrated into the genome, it is preferred that these expression cassettes contain a promoter which is repressed by absence of the synthetic compound, e.g. a Tet-off promoter, in order to allow a MHC-expression dependant healthy development of the animals in the absence of the synthetic compound with no activity of these expression cassettes, so that after transplantation of this tissue into a recipient, activity of the expression cassettes for transcription of inhibitory RNA, and accordingly for downregulation expression of MHC I and of MHC II in the tissue can be induced by administration of the synthetic compound, e.g. tetracycline or doxycycline for use in the treatment of HvG in a recipient of the tissue. The Tet-operator element can be SEQ ID NO: 13. Alternatively, a promoter which is repressed by the presence of the synthetic compound, e.g. a Tet-on promoter, can be used, allowing a MHC-expression dependant healthy development of the animals in the presence of the synthetic compound with no activity of these expression cassettes, so that after transplantation of this tissue into a recipient, activity of the expression cassettes for transcription of inhibitory RNA, and accordingly for downregulation expression of MHC I and of MHC II in the tissue can be induced by the absence of the synthetic compound, e.g. tetracycline for use in the treatment of HvG in a recipient of the tissue. In either case, the level of expression can be adjusted to the level needed to prevent detrimental immune reactions and enable beneficial Treg cell immune reactions as described. Generally, such non-human animals are genetically manipulated to contain, by somatic genetic manipulation or preferably in their germline, nucleic acid constructs which encode inhibitory RNA under the control of a conditional promoter system which can be regulated in a dose-dependent manner by the presence of a synthetic compound, wherein inhibitory RNA under the control of the conditional promoter is arranged to downregulate expression of the MHC I by at least 50% to up to 90%, wherein inhibitory RNA under the control of the conditional promoter is arranged to downregulate expression of MHC II by at least 50% to up to 90%, wherein the downregulation of the MHC I and/or of the MHC II is in relation to its expression in wild-type tissue, and wherein the nucleic acid construct, optionally under the control of the conditional promoter, additionally expresses at least one complement inhibitory molecule. Such non-human animals can e.g. be genetically manipulated to contain nucleic acid constructs of the invention.

The tissue for use as a transplant is accordingly genetically manipulated to expresses their original MHC I molecules and/or original MHC II molecules at a residual expression level of at maximum 40%, at maximum 30%, more preferably at maximum 20% to 10% or 20% to 15% of the expression level of MHC I and respectively of MHC II molecules in relation to the wild-type tissue or in relation to the expression level in the recipient of the transplant, wherein the original MHC I molecules of the tissue are identical or allogeneic or xenogeneic and/or their original MHC II molecules of the tissue are identical or allogeneic or xenogeneic to the MHC I and MHC II molecules of a recipient of the transplant. Therein, the tissue for use as a transplant is genetically manipulated to downregulate expression of its original MHC I by at least 50% to up to 90%, leaving a residual expression of at maximum 50% to 10%, and preferably genetically manipulated that the expression of the original MHC I is downregulated by at least 60%, more preferably by at least 70%, more preferably by 80% up to 90% or up to 85%, leaving a residual expression of the original MHC I of at maximum 40%, at maximum 30%, more preferably at maximum 20% to 10% or 20% to 15%, and the tissue for use as a transplant is genetically manipulated that the expression of its original MHC II is downregulated by at least 50%, e.g. by at least 60%, e.g. genetically manipulated that expression of its original MHC II is downregulated by up to 90%, preferably downregulated by at least 70%, more preferably downregulated by at least 80%, e.g. downregulated by up to 90% or by up to 85%, leaving a residual expression of the wild-type MHC II of at maximum 50%, e.g. of at maximum 40%, e.g. of at least 10%, preferably of at maximum 30%, more preferably of at maximum 20%, e.g. a residual expression of at least 10% or at least 15%, wherein the downregulation of the original MHC I and/or of the original MHC II of the tissue for use as a transplant is in relation to its expression in wild-type tissue or is in relation to the expression level of MHC I and MHC II, respectively, in the recipient of the transplant, and additionally expressing at least one complement inhibitory molecule on the tissue, which complement inhibitory molecule is at least one of CD46, CD55 and CD59, preferably two of these, more preferably expression of CD46 and of CD55 and of CD59. Generally preferred, in the tissue for use as a transplant, which tissue expresses MHC I and MHC II molecules, the expression of the MHC I is downregulated by at least 50% to up to 90% and the expression of MHC I is downregulated by at least 60%. Generally herein, the term allogeneic MHC I and MHC II may comprise identical MHC I and identical MHC II, respectively, to the MHC I and MHC II of the recipient. Preferably, the tissue is genetically manipulated to express at least one complement inhibitory molecule. Herein, the wild-type tissue can also be referred to as original tissue, e.g. the tissue prior to genetic manipulation. Generally preferably, the tissue for use as a transplant is free from genetic manipulation resulting in expression of additional or different MHC I or MHC II molecules. Accordingly, the tissue for use as a transplant, e.g. for use in a method of treatment, is genetically manipulated for downregulation of its original MHC I and/or its original MHC II, and is devoid of genetic manipulation expressing additional MHC I and/or MHC II, e.g. devoid of expression cassettes encoding MHC I and/or MHC II of the recipient of the transplant, and/or devoid of expression of HLA-G.

The invention is now described by way of examples with reference to the figures, which show in

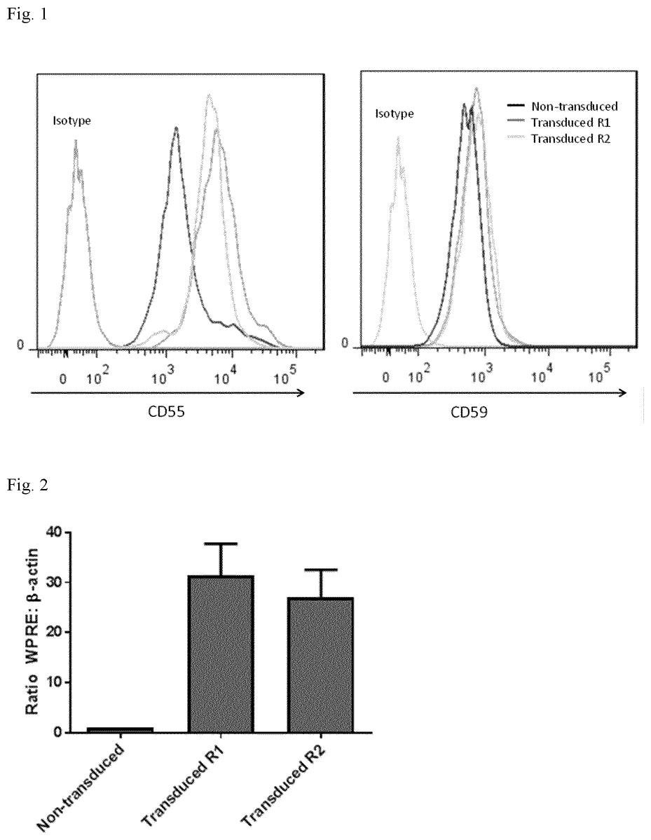

FIG. 1 expression levels of CD55 and of CD59 in cells that were lentivirally transduced,

FIG. 2 PCR analytical results for integration of expression cassettes in tissue cells,

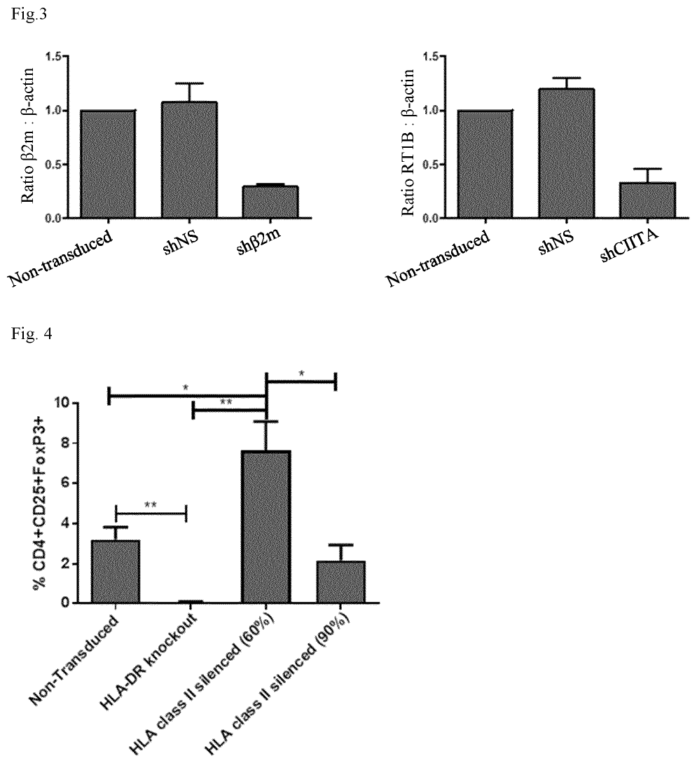

FIG. 3 transcript levels of f32m and of MHC II in tissue cells expressing inhibitory RNA,

FIG. 4 FACS results detecting Treg cells expanded in presence of cells with downregulated expression of MHC I and MHC II in comparison to MHC II knockout cells,

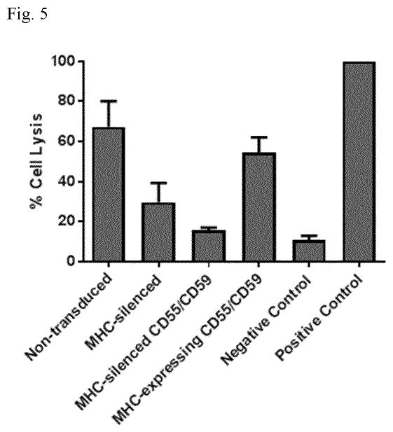

FIG. 5 the percentage of lysed cells of tissue according to the invention which contain expression cassettes for different complement inhibitory molecules.

### EXAMPLE 1

### Genetic Manipulation of Xenogeneic Pig Islet Cells for use as Transplant

As an example for a tissue expressing xenogeneic MHC I and MHC II molecules, pig islet cells were used. For genetic manipulation of these xenogeneic cells a lentiviral vector backbone was used, which contained an expression cassette encoding according to the invention an siRNA (SEQ ID NO: 6) specific for the transcript of the Sus scrofa J32-microglobulin gene (SEQ ID NO: 30) and/or an expression cassette encoding an siRNA (SEQ ID NO: 8) specific for the transcript of the class II transactivator gene (CIITA) (SEQ ID NO: 31), preferably both expression cassettes. The inhibitory RNAs were designed and sufficient for a downregulation of MHC class I and MHC class II by 80% but not more than 90%. Following transplantation into mice as transplant recipients it was found that no cellular immunogenic response was elicited in the recipient, and no T-cell-mediated graft rejection occurred.

It was found that antibodies directed against the xenogeneic MHC I and MHC II molecules can occur, but the formation of anti-MHC I xeno-antibodies and of anti-MHC II xeno-antibodies would not result in antibody-mediated graft rejection when the suppression, i.e. downregulation, of the xenogeneic MHC I and of the xenogeneic MHC II in the tissue was by at least 80% compared to expression of the xenogeneic MHC I and MHC II, respectively, determined for wild-type pig islet cells, e.g. determined for cells treated in parallel but without genetic manipulation. For improved survival of the tissue, it was transduced additionally by a lentiviral vector which contained expression cassettes encoding complement inhibitors CD55 (SEQ ID NO: 17) and CD59 (SEQ ID NO: 19). It was found that a less intense downregulation of expression of the xenogeneic MHC I and MHC II does not result in antibody-mediated graft rejection when the xenogeneic cells expressed higher levels of complement inhibitors.

### EXAMPLE 2

### Genetic Manipulation of Allogeneic Kidney for use as Transplant

As an example for a tissue expressing allogeneic MHC I and MHC II molecules, a rat kidney that was genetically manipulated according to the invention was used as an allogeneic transplant in a recipient rat.

For genetic manipulation of the exemplary tissue, a nucleic acid construct was made which contained expression cassettes for a shRNA encoding SEQ ID NO: 9, which according to the invention is specifically targeting the rat 02-microglobulin mRNA (SEQ ID NO: 26), and an shRNA encoding SEQ ID NO: 10, according to the invention specifically targeting the rat MHC class II transactivator (CIITA) (SEQ ID NO: 27), as well as the coding sequences for the complement inhibitors CD55 (SEQ ID NO: 18) and CD59 (SEQ ID NO: 20). The shRNA targeting the 02-microglobulin mRNA causes silencing of MHC class I in a specific and selective manner without exceeding a level of suppression above 80% compared to untreated, i.e. wild-type, cells and the shRNA targeting the MHC class II transactivator (CIITA) causes silencing of MHC class II in a specific and selective manner without exceeding a level of suppression of 70% compared to untreated cells. The nucleic acid construct further contained an expression cassette for Luciferase, suitable for labelling cells in which the nucleic acid construct is integrated.

This nucleic acid construct in addition to shRNA targeting MHC transcripts optionally contained the coding sequences for CD55 and CD59. The nucleic acid constructs with or without the additional coding sequences for CD55 and CD59 were packaged in lentiviral particles using packaging cells, were concentrated by centrifugation at 30 000×g for 8 h and resuspended in the preservation solution. The viral particle titer was determined using p24 ELISA and the lentiviral vector particles were stored was at −80° C.

As an example for an allogeneic isolated vascularized tissue, the explanted rat kidneys of LEW1U.RT1 rats were flushed with preservation solution (composed of Custodiol) containing 80 IE heparin at a temperature of 4° C. immediately after explantation. The flushed rat kidneys were connected to an ex vivo perfusion system, warmed up to 37° C. and perfused for 2-3 h with the vector encoding the shRNA sequences directed against MHC I and MHC II, or with the vector encoding both the shRNA sequences directed against MHC I and MHC II and expression cassettes encoding CD55 and CD59 sequences, using Custodiol supplemented with 8 μg/mL Protamin sulphate and 4mg Urbason. 10⁹-10¹⁰ lentiviral particles were used per kidney. During perfusion the temperature was maintained at 37° C. and a flow rate of 10-14 mL/min was used. After perfusion with the vector, the perfusion solution was replaced with Custodiol containing 3-6mL of heparinized peripheral rat blood and the kidneys were perfused for 20-30 min to remove the free residual vector. Afterwards, the kidneys were washed with fresh Custodiol solution and cooled down to 4° C.

For analyses, kidney tissue was taken from different regions and digested with collagenase VI and dispase (both obtained from Sigma). At 96 h after the addition of the lentiviral vector to the second perfusion solution, endothelial cells of the kidney macrovasculature and micro-vasculature after labelling with anti-CD31 antibody were isolated using paramagnetic beads (Dyna-beads, obtained from Thermo Fisher) for magnetic separation. In parallel, endothelial cells were isolated from a non-transduced kidney which was treated in the same way, except that no viral particle was added. Analyses showed that endothelial cells were isolated at purities above 70%. Endothelial cells were cultivated in gelatine-coated tissue culture plates. Five days after perfusion with the lentiviral vector the MHC silencing effect and the levels of expression of CD55 and CD59 were evaluated by flow cytometric analyses.

At 24 h after addition of the viral particles to the second perfusion solution, luciferase activity was detected in the culture supernatant. The levels of this bioluminescence increased during the 96 h of cultivation. Remarkably, cells from the kidneys showed to be transduced, indicating integration and activity of the nucleic acid construct in the entire organ endothelium. Luciferase activity was measured after addition of luciferase substrate (Furimazine, Promega).

FIG. 1 depicts the expression levels of CD55 and CD59 in rat kidney endothelial cells isolated from two different regions designated R1 and R2 of the kidney perfused with the lentiviral vector encoding for CD55 and CD59 in comparison to the native levels expressed in a kidney perfused without vector (non-transduced). The result shows that lentiviral particles provided for stable and permanent integration the nucleic acid construct including the expression cassettes into the genome of the vascularized tissue.

FIG. 2 depicts the results of real-time PCR using DNA that was isolated from the cultivated rat endothelial cells and primers that were specific for the woodchuck hepatitis virus posttranscriptional element WPRE, which is present in the nucleic acid construct. The result shows the high levels of WPRE, respectively of the nucleic acid construct according to the invention, in the endothelial cells treated by the method of the invention, and background levels only in the endothelial cells from the kidneys that were treated in parallel but without addition of the lentiviral vector

Isolated cells were cultured in EBM-2 endothelial cell growth medium (obtained from Lonza). Endothelial cells were stimulated with IFN-γ (100ng/mL) for 48 h. It was found that endothelial cells from the microvasculature of the non-transduced parallel treatment were able to up-regulate MHC class I (RT1-A) and MHC class II (RT1-B) transcript levels (p<0.001) upon exposure to IFN-65 , but microvascular endothelial cells from kidneys treated with the nucleic acid construct according to the invention, contained in the lentiviral particle, upon IFN-γ stimulation did not show a significant increase in transcript levels for (32-microglobulin and RT1-B. These results demonstrate that the method of the invention effectively silences expression of MHC class I and of MHC class II in the vascularized tissue and leads to an expression of the transgenes CD55 and CD59. Also, no ischemic damage was found, which could be caused by undersupply with oxygen.

FIG. 3 depicts the levels detected by RT-PCR for transcripts of (32-microglobulin and for transcripts of MHC class II (RT1-B) after stimulation with IFNy for 48 h in the cultivated endothelial cells. As a negative control, non-transduced cells were used and cells that were transduced with a vector encoding a non-binding non-specific shRNA (shNS). Transcript levels are normalized to levels of transcripts for (3-actin (***p<0.001), also detected by RT-PCR. These results show that the nucleic acid construct encoding shRNA specific for transcripts of (32-microglobulin, respectively for transcripts of CIITA, effectively downregulate expression of (32-microglobulin and of CIITA, and hence downregulate expression of MHC I and MHC II.

FIG. 4 depicts the analytical result of FACS for Treg cells (CD4+CD25+FoxP3+). The result shows that frequencies of Treg cells that were expanded in the presence of cells containing an expression cassette for shRNA directed against MHC II transcripts for downregulation by 60% (HLA class II silenced (60%)) or by 90% (HLA class II silenced (90%)), are higher than the frequencies of Tregs in the presence of MHC class II knockout cells (HLA-DR knockout, 100% downregulated) and in the moderate downregulation of MHC class II transcripts by 60% even higher than with non-transduced cells. HLA-DR knockout single donor HUVEC cells or HLA class II silenced single donor HUVEC cells that were downregulated for up to 60% or up to 90% were co-cultured in presence of PBMCs in RPMI medium supplemented with 5% AB serum and 100U/mL IL-2 and 20ng TGF-(3. Non-transduced cells were used as controls (Non-Transduced).

For allogeneic transplantation, the kidneys from explanted kidneys of LEW1U.RT1 donor rats were used as allogeneic transplants in LEW.RT1 rats as recipients. The right kidney was perfused and genetically manipulated as described above and then was transplanted in rat LEW.RT1 recipients in an orthotopic manner. During the operation the animals are maintained on a thermoplate to maintain the body temperature constant. The recipient was ventilated via a respiratory mask. For anaesthesia, Isofluran was administered. After laparotomy the right kidney was resected and the vessels clamped. The genetically modified or unmodified control donor kidneys, respectively, was meanwhile cooled in the ex vivo perfusion system. The kidney was removed from the ex vivo perfusion system, flushed with fresh cold perfusion solution and prepared for implantation. The kidney was then transplanted using standard techniques. After discontinuation of anesthesia and removal of the respiratory mask, the animals were put into boxes provided with drinking water. The animals were regularly controlled for signs of rejection and cytokine levels such as IP-10, MCP-1 or RANTES were monitored. In the first 14 days after transplantation the animals received a mild immunosuppression (10mg/kg body weight Cyclosporin/day). Animals that were transplanted with a genetically modified organ did not show clinical, immunological or morphological signs of immunological organ rejection within an observation period of 90 days, whereas animals that were transplanted with an unmodified kidney rejected the graft at the latest 30 days after transplantation.

FIG. 5 depicts the percentage of lysed cells which contain expression cassettes for different complement inhibitory molecules. The results show that the overexpression of CD55 and CD59 in MHC-silenced rat endothelial cells (LEW.1W) substantially decreases antibody-mediated cytotoxicity nearly to the level of the negative control. Rat endothelial cells (ECs) were incubated with serum of an immunized rat containing anti-RT1u antibodies and complement. MHC-silenced ECs overexpressing CD55 and CD59 were best protected against complement-dependent antibody-mediated cell lysis. Only MHC-silenced ECs showed an improved protection compared to non-MHC-silenced only CD55 and CD59 transgenes expressing ECs. The data was normalized to the positive control. The negative control used serum from the EC donor (autologous control).

This example shows that the transplantation of an allogeneic kidney does not induce an immunogenic cellular reaction by the recipient and does not induce a T-cell and B-cell-mediated rejection (HvG), which kidney was genetically manipulated by retroviral transduction to contain expression cassettes for downregulating expression of its MHC I and MHC II by at least 80%, leaving a residual expression of at most 20%, compared to wild-type expression, and to contain expression cassettes for expression of complement inhibitors CD55 an CD59.

1. Tissue for use as a transplant, which tissue expresses allogeneic or xenogeneic MHC I and/or allogeneic or xenogeneic MHC II molecules, wherein the expression of the allogeneic or xenogeneic MHC I is downregulated by at least 50% to up to 90%,
wherein the expression of the allogeneic or xenogeneic MHC II is downregulated by at least 50% to up to 90%, wherein the downregulation of the allogeneic or xenogeneic MHC I and/or of the allogeneic or xenogeneic MHC II is in relation to its expression in wild-type tissue,
and wherein the tissue additionally expresses at least one complement inhibitory molecule on the tissue.

2. Tissue for use as a transplant according to claim 1, characterized in that the expression of the allogeneic or xenogeneic MHC I is downregulated by 80% up to 90%, and wherein the expression of the allogeneic or xenogeneic MHC II is downregulated by at least 80% to up to 90%.

3. Tissue according to claim 1, wherein the expression of the allogeneic or xenogeneic MHC I and/or the expression of the allogeneic or xenogeneic MHC II is downregulated by the tissue being genetically manipulated to contain at least one nucleic acid construct comprising at least one expression cassette encoding inhibitory RNA interfering with at least one transcript encoding the allogeneic or xenogeneic MHC I molecule and/or encoding the allogeneic or xenogeneic MHC II molecule.

4. Tissue according to claim 1, wherein the expression of the allogeneic or xenogeneic MHC I is downregulated by the tissue being genetically manipulated to contain a nucleic acid construct comprising at least one expression cassette encoding an siRNA which hybridizes to the transcript of the 02-microglobuline gene (β2m), and/or encoding an siRNA which hybridizes to the transcript of at least one, or two, preferably for transcripts of all three heavy chains of HLA-A, HLA-B and HLA-C molecules or SLA-1, SLA-2 and SLA-3 molecules.

5. Tissue according to claim 1, wherein the expression of the allogeneic or xenogeneic MHC II is downregulated by the tissue being genetically manipulated to contain a nucleic acid construct comprising at least one expression cassette encoding an siRNA which hybridizes to transcripts of at least one of the alpha chain and/or beta chain of HLA-DR, HLA-DQ and/or of HLA-DP or SLA-DR and/or SLA-DQ, and/or by encoding an siRNA which is specific for transcripts of the class II transactivator gene.

6. Tissue according to claim 1, wherein complement inhibitory molecule is selected from CD46, CD55 and CD59.

7. Tissue according to claim 1, comprising use for or excluding or reducing immunosuppressants for permanent use in the treatment of HvG disease.

8. Tissue according to claim 1, wherein tissue is a portion of an organ or of an extremity or a complete organ or extremity.

9. Tissue according to claim 1, wherein the expression of the allogeneic or xenogeneic MHC I is downregulated by 80% up to 90% in relation to the expression level of MHC I in the wild-type tissue, and wherein the expression of the allogeneic or xenogeneic MHC II is downregulated by at least 80% to up to 90% in relation to the expression level of MHC I in the wild-type tissue.

10. Tissue according to claim 1, wherein the expression of the allogeneic or xenogeneic MHC I is downregulated by 80% up to 90% in relation to the expression level of MHC I in the recipient of the transplant, and wherein the expression of the allogeneic or xenogeneic MHC II is downregulated by at least 80% to up to 90% in relation to the expression level of MHC I in the recipient of the transplant.

11. Tissue according to claim 1, wherein the tissue consists of or comprises single cells, e.g. islet cells, a portion of an organ or a complete organ, which organ is e.g. a kidney, liver, heart, lung, pancreas, skin, or an extremity.

12. Tissue according to claim 1, being devoid of expression of an additional MHC I molecule and/or MHC II molecule.

13. Tissue according to claim 1, being devoid of expression of HLA-G.

14. Non-human mammalian animal which is genetically manipulated to comprise tissue according to claim 1.

15. Non-human animal according to claim 14, wherein the animal is genetically manipulated to contain, in its germline or by somatic genetic manipulation, nucleic acid constructs which encode inhibitory RNA under the control of a conditional promoter system which can be regulated in a dose-dependent manner by the presence of a synthetic compound, wherein inhibitory RNA under the control of the conditional promoter is arranged to downregulate expression of the MHC I by at least 50% to up to 90%,
wherein inhibitory RNA under the control of the conditional promoter is arranged to downregulate expression of MHC II by at least 50% to up to 90%, wherein the downregulation of the MHC I and/or of the MHC II is in relation to its expression in wild-type tissue,
and wherein the nucleic acid construct, optionally under the control of the conditional promoter, additionally expresses at least one complement inhibitory molecule.

16. Non-human animal according to claim 15, being devoid of expression of HLA-G.

17. Process for producing a tissue expressing an MHC I and an MHC II for use as a transplant, comprising providing an isolated tissue with a nucleic acid construct that comprises at least one expression cassette encoding at least one siRNA set up to downregulate the expression of the MHC I by at least 50% to up to 90% in relation to its expression in wild-type tissue, and comprises at least one expression cassette encoding at least one siRNA set up to downregulate the expression of the MHC II by at least 50% to up to 90% in relation to its expression in wild-type tissue, and comprising providing the isolated tissue with a nucleic acid construct containing an expression cassette encoding at least one complement inhibitory molecule.

18. Process according to claim 17, wherein the MHC I and MHC II are allogeneic or xenogeneic for a recipient of the transplant.

19. Process according to claim 1, wherein no genetic manipulation of the isolated tissue is performed that would result in expression of HLA-G.

20. Method of treatment of a patient by transplanting a tissue of claim 1 into the patient.

21. Nucleic acid construct for use in the treatment of HvG disease, the nucleic acid construct comprising at least one expression cassette encoding an siRNA set up to downregulate expression of an MHC I of a tissue by at least 50% to up to 90%, at least one expression cassette encoding an siRNA set up to downregulate expression of an MHC II of a tissue by at least 50% to up to 90%, and expression cassettes encoding at least one complement inhibitory molecule.

22. Nucleic acid construct according to claim 21, comprising at least two nucleic acid molecules.

23. Nucleic acid construct according to claim 21, having one nucleic acid molecule.
